(19) United States
(12) Patent Application Publication
(10) Pub. No.: US 2026/0272618 A1
(43) Pub. Date: Sep. 17, 2026

(54) Horizontal Wing Scanflag for Full Arch Scanning and Enhanced Matching Accuracy in Intraoral Scanning Systems

(71) Applicant: Abdelalim Sameh Sultan, Jeddah (SA)

(72) Inventor: Abdelalim Sameh Sultan, Jeddah (SA)

(21) Appl. No.: 19/080,880

(22) Filed: Mar. 16, 2025

(51) Int. Cl. A61C 9/00; A61B 1/00; A61B 1/24

(52) U.S. Cl. CPC A61C 9/0053; A61B 1/00172; A61B 1/24

(57) ABSTRACT

The invention provides a horizontal wing scan flag for use in intraoral scanners, designed to simplify and enhance the process of capturing full arch scans and accurately matching scan bodies. Traditional scan flags rely on vertical matching, which can be challenging to scan and less precise, or on lateral flags that still incorporate vertical elements or require additional manual adjustments in design software. The horizontal wing scan flag utilizes its horizontal lateral part to directly match the scan body during scanning and in CAD/CAM design software, eliminating the need for time-consuming replacements or adjustments. This design ensures improved accuracy, streamlined workflows, and reduced scanning time. The invention is particularly advantageous for hard-to-scan cases and full arch scanning, setting a new standard for intraoral scanner compatibility and usability.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

This invention relates to the field of digital dentistry and intraoral scanning systems, particularly to the design and use of scanflags for capturing full arch scans and accurately registering scanbodies. Specifically, it introduces a horizontal wing scanflag that improves the ease, precision, and efficiency of matching scanbodies during the design phase in dental CAD/CAM software, addressing limitations of existing vertical and lateral scanbody matching techniques.

### Description of the Related Art

The dental industry has witnessed a paradigm shift with the advent of digital dentistry, particularly the use of intraoral scanners for creating precise digital impressions. These scanners have revolutionized workflows, enhancing speed, accuracy, and patient comfort compared to traditional methods. Central to these workflows is the use of scanbodies, or “scanflags,” which serve as reference markers during the scanning process. Despite their critical role, conventional scanflags often exhibit design inefficiencies that hinder their effectiveness in complex cases, such as full-arch scans or difficult-to-access regions of the oral cavity. This document presents an innovative horizontal wing scanflag that addresses these challenges and redefines the intraoral scanning process.

Intraoral scanners and scanbodies have become indispensable tools in restorative and orthodontic procedures. Precise digital impressions directly impact the accuracy of prosthetics, crowns, bridges, and implants, ultimately influencing patient outcomes and satisfaction. An efficient and accurate scanbody design is crucial for ensuring seamless integration between scanning and design software. However, the limitations of current designs—such as dependency on vertical alignment parts—have hindered their usability in challenging scenarios. Developing a scanflag that simplifies the process while enhancing accuracy is essential for advancing digital dental workflows.

The global intraoral scanner market was valued at approximately $900 million in 2023 and is expected to grow at a compound annual growth rate (CAGR) of over 10% by 2030. This growth is driven by increasing demand for digital dentistry, heightened awareness of minimally invasive treatments, and advancements in dental CAD/CAM technologies. Despite this growth, surveys indicate that over 30% of dental practitioners still face challenges with scan accuracy in full-arch cases, while 45% report time inefficiencies due to repeat scans or manual adjustments. These figures underscore the pressing need for improved scanbody solutions tailored to modern demands.

Several companies and researchers have introduced advancements to improve scanbody designs. Some have incorporated lateral flags to complement vertical parts, while others have explored hybrid designs that can be individually modified during the scanning process. These innovations represent meaningful progress, particularly in enhancing scan accessibility and reducing hardware adjustments. However, they often involve complex workflows, such as replacing horizontal parts before integrating scans into design software. While such solutions are promising, they still fall short of delivering a comprehensive and user-friendly solution for full-arch or hard-case scenarios.

Despite advancements, users still encounter significant challenges in current scanbody designs. The reliance on vertical parts for matching creates scanning difficulties in confined or obstructed areas of the oral cavity, leading to inaccuracies and incomplete scans. Moreover, solutions involving lateral flags often require manual replacement or modification of parts, which introduces additional steps and potential errors in the workflow. These issues not only compromise accuracy but also increase time and labor, creating bottlenecks in clinical and laboratory settings.

The proposed horizontal wing scanflag represents a transformative innovation in intraoral scanning technology. This design eliminates reliance on vertical parts for matching and instead leverages the horizontal lateral section as the primary matching interface. The horizontal wing is engineered to provide a clear and accessible surface for accurate digital capture, even in challenging scenarios such as full-arch cases. Additionally, the design integrates seamlessly with existing dental CAD/CAM software, removing the need for manual part replacement or modification before processing scans.

This invention addresses the key limitations faced by dental professionals using conventional scanflags. By utilizing a horizontal lateral matching mechanism, it ensures greater scanning accuracy and reduces the likelihood of errors caused by inaccessible vertical components. Furthermore, the elimination of time-consuming part modifications streamlines workflows, allowing practitioners to achieve faster and more reliable results. The horizontal wing scanflag is particularly well-suited for complex cases, ensuring consistent and precise outcomes in full-arch and hard-to-reach regions. By filling this critical market gap, the invention stands to redefine the standard for intraoral scanning, providing a practical and efficient solution that meets the evolving demands of digital dentistry.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The present invention seeks to improve prior techniques and provide an advanced design and methodology to improve the accuracy of scanbody matching by utilizing the horizontal lateral portion of the scanflag, reducing errors common in vertical matching techniques.

The invention also aims to enhance the efficiency of intraoral scanning workflows by eliminating the need for manual adjustments or replacement steps in CAD/CAM software.

A further objective of the present invention is to reduce the time required for full arch and hard case scans, providing faster and more reliable outcomes.

It is also an object of the invention to simplify the scanning process for dental professionals, especially in capturing challenging areas of the oral cavity.

It is further the objective of the invention to enable seamless integration with existing CAD/CAM software, ensuring a smooth transition into digital workflows.

It is also the objective of the invention to provide a reusable, durable scanflag that is cost-effective for clinical applications.

It is also the objective of the invention to enhance patient comfort during intraoral scanning by reducing the need for repetitive or prolonged scanning procedures.

It is also the objective of the invention to minimize post-scan processing time by directly capturing data in a form ready for use in design software.

It is further the objective of the invention to increase the reliability of dental restorations and prosthetics, thanks to the improved precision of the scan data.

The invention aims to support advancements in digital dentistry technologies, paving the way for future innovations in intraoral scanning and design systems.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 shows the complete side view of the proposed assembly incorporating the embodiments of the invention.

FIG. 2 shows top view of the nasal aspirator incorporating the embodiments of the invention.

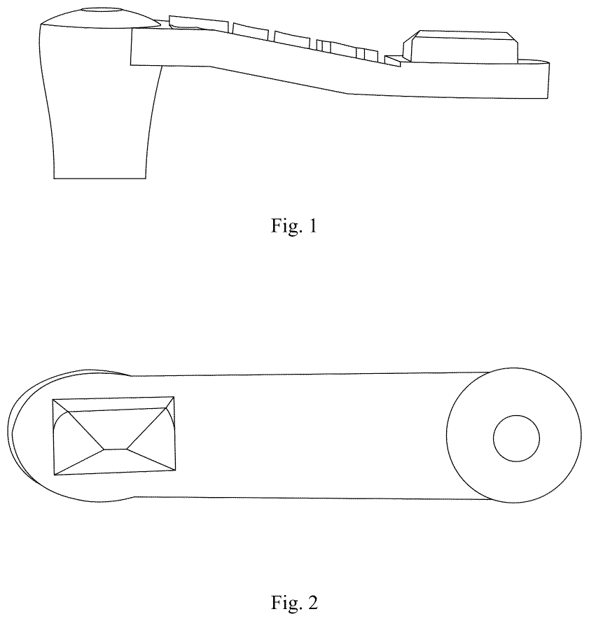

The apparatus and method components have been represented where appropriate by conventional symbols in the drawings, showing only those specific details

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present invention relates to the field of intraoral scanning technology, specifically to scanbody attachments for dental applications. More particularly, it pertains to an innovative horizontal wing scanflag designed to improve the accuracy and efficiency of full-arch and complex case scanning, providing a novel matching mechanism for integration with dental design software.

Intraoral scanners have revolutionized dental practices by offering precise digital impressions. However, current scanbody designs, commonly referred to as “scanflags,” exhibit significant limitations. Most existing scanflags rely on vertical alignment parts for matching purposes. This approach often results in suboptimal accuracy, especially in challenging cases involving full arches or hard-to-reach areas within the oral cavity. Companies that incorporate lateral flags either revert to vertical parts for matching or rely on a labor-intensive process of individually replacing horizontal components prior to software integration. Such methods are not only time-consuming but may also introduce errors, compromising overall precision and workflow efficiency. Therefore, there exists a critical need for a more effective and reliable solution.

As per its preferred embodiments, the present invention introduces a horizontal wing scanflag designed specifically to address the deficiencies of current systems. Unlike traditional scanflags that depend on vertical parts for matching, this invention utilizes the horizontal lateral portion of the scanbody for precise alignment and matching in dental design software. This novel approach simplifies scanning procedures, enhances matching accuracy, and reduces the time required for workflow processes. The horizontal wing design is particularly effective for full-arch scans and complex cases, where conventional methods struggle to deliver consistent results.

As per its further embodiments, the horizontal wing scanflag comprises a horizontal lateral part strategically engineered to function as the primary matching interface. When used in conjunction with intraoral scanners, the horizontal wing provides a clear and unobstructed surface that is easier to capture digitally. This design eliminates the dependency on vertical components, which are prone to inaccuracies due to limited visibility and scanning difficulties in confined oral spaces. The horizontal wing seamlessly integrates with existing dental design software, ensuring precise alignment and accurate reconstruction of the scanned area.

As per its further embodiments, the invention offers several advantages over traditional scanflags. Firstly, the horizontal lateral part allows for a more straightforward scanning process, reducing the need for multiple rescans and operator adjustments. Secondly, the enhanced accuracy of the horizontal wing eliminates the need to replace or modify scanbody parts before software integration, streamlining the workflow and minimizing potential errors. Thirdly, the horizontal design is inherently more ergonomic for scanning full arches and hard cases, offering a significant improvement in efficiency for dental practitioners.

As per its further embodiments, the invention is particularly beneficial for dental professionals performing complex restorative or orthodontic procedures. The horizontal wing scanflag ensures high-precision digital impressions for full-arch cases, challenging implant placements, and other intricate dental applications. It also facilitates seamless integration with computer-aided design (CAD) software, making it an invaluable tool in modern digital dentistry.

As per its further embodiments, the existing products that incorporate lateral flags still rely on vertical parts for matching or involve manually replacing horizontal components. These approaches are inherently limited in their accuracy and time efficiency. The present invention is unique in its ability to utilize the horizontal lateral part as the sole matching interface, thereby offering superior performance and reliability compared to similar products in the market.

As per its further embodiments, the horizontal wing scanflag is constructed using a durable and biocompatible material suitable for repeated use in intraoral environments. The lateral wing section features a textured or patterned surface to optimize digital scanning accuracy. The design ensures compatibility with a wide range of intraoral scanners and dental design software platforms, making it a versatile addition to any dental practice.

As per its further embodiments, the horizontal wing scanflag represents a significant advancement in intraoral scanning technology. By addressing the limitations of current scanbody designs and introducing a horizontal matching mechanism, this invention enhances accuracy, reduces workflow complexity, and improves overall efficiency in dental procedures. This innovative approach sets a new standard for digital impression techniques, providing dental professionals with a reliable and user-friendly solution for full-arch and hard case scanning challenges.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

We claim:

1. A horizontal wing scanflag for intraoral scanners, comprising:
a. a horizontal lateral portion specifically designed to facilitate direct and accurate matching of scanbodies;
b. an integrated full arch scanning capability for complex dental cases; and
c. compatibility with intraoral scanners and CAD/CAM software for improved digital workflows.
The horizontal wing scanflag of claim 1, wherein the horizontal portion includes textured surfaces to enhance optical recognition during scanning.
The horizontal wing scanflag of claim 1, wherein the device features color-coded indicators for ease of positioning and alignment.
The horizontal wing scanflag of claim 1, wherein the lateral portion incorporates anti-reflective materials to reduce scanning errors.
The horizontal wing scanflag of claim 1, wherein the flag design is modular, allowing replacement of individual components without discarding the entire unit.
The horizontal wing scanflag of claim 1, wherein the material is heat-sterilizable for repeated use in clinical settings.
The horizontal wing scanflag of claim 1, wherein the design includes unique serial numbers for traceability and quality control and it provides enhanced data to capture for areas that are typically difficult to scan with vertical flags.

2. A method of using the horizontal wing scanflag in digital dentistry workflows, comprising:
a. attaching the horizontal wing scanflag to a scanbody;
b. performing full arch or complex case scans with an intraoral scanner; and
c. directly utilizing the captured lateral data for automatic matching in CAD/CAM software without manual adjustments.
The method of claim 2, wherein the scanning process includes real-time feedback for ensuring optimal alignment.
The method of claim 2, wherein data captured from the horizontal wing scanflag is integrated into a preloaded library for streamlined design.
The method of claim 2, wherein the scanning technique reduces average scanning time by 30% compared to traditional methods.
The method of claim 2, wherein the method includes calibration of the scanner to maximize compatibility with the horizontal wing scanflag.
The method of claim 2, wherein the captured data undergoes automatic error correction in the CAD/CAM software.
The method of claim 2, wherein the scanflag can be detached and repositioned without loss of data integrity.
The method of claim 2, wherein the scanflag supports multi-surface scanning in a single session for comprehensive data capture.

3. An intraoral scanning system incorporating the horizontal wing scanflag, comprising:
a. a horizontal wing scanflag configured to match scanbodies directly using the horizontal lateral portion;
b. an intraoral scanner with optimized software for recognizing and aligning horizontal data; and
c. a CAD/CAM system for processing and utilizing the scan data in digital workflows.
The intraoral scanning system of claim 3, wherein the scanner includes a real-time alignment tool for horizontal matching.
The intraoral scanning system of claim 3, wherein the CAD/CAM software includes templates specifically designed for horizontal wing scanflag integration.
The intraoral scanning system of claim 3, wherein the scanner offers wireless connectivity to transmit captured data directly to the CAD/CAM system.
The intraoral scanning system of claim 3, wherein the scanner supports integration with cloud-based dental libraries for extended functionality.
The intraoral scanning system of claim 3, wherein the system includes a notification system to alert users about incomplete or suboptimal scans.
The intraoral scanning system of claim 3, wherein the scanner is compatible with multi-platform operating systems for greater flexibility.
The intraoral scanning system of claim 3, wherein the system includes a post-processing feature to refine scan data for better prosthetic outcomes.
